(19) United States
(12) Patent Application Publication
(10) Pub. No.: US 2026/0266816 A1
(43) Pub. Date: Sep. 10, 2026

(54) ANALYSIS DEVICE

(71) Applicant: CANON KABUSHIKI KAISHA, Tokyo (JP)

(72) Inventors: TAKAYUKI KANAZAWA, Kanagawa (JP); TAKESHI YAMAMOTO, Kanagawa (JP); MAKOTO FUKATSU, Shizuoka (JP); KEIJI MIYAZAKI, Tokyo (JP); MASANORI TANAKA, Kanagawa (JP); JUN MIURA, Kanagawa (JP); FUKA ENOKIDO, Kanagawa (JP)

(21) Appl. No.: 19/555,527

(22) Filed: Mar. 3, 2026

(30) Foreign Application Priority Data
Mar. 4, 2025 (JP) 2025-033813

(51) Int. Cl. G01N 33/543

(52) U.S. Cl. CPC G01N 33/54388

(57) ABSTRACT

The present disclosure provides an analysis device including a first sheet and a second sheet which is laminated below the first sheet, the first sheet including: a first channel wall formed by filling pores of a porous substrate with a hydrophobic material; and a first channel formed by being bound by the first channel wall. An upper surface of the first sheet is defined as a first surface and a lower surface of the first sheet is defined as a second surface and a cross-sectional area of the first channel, which is located in a direction which is parallel to the first surface and the second surface, is defined as a channel area A, and the first channel has a region A formed so that the channel area A decreases from the first surface toward the second surface.

### BACKGROUND

### Field of the Technology

The present disclosure relates to an analysis device.

### Description of the Related Art

### Microchannel Technologies

In recent years, development of a microchannel device (also referred to as an analysis device in the present disclosure) that can perform analysis in biochemistry in one chip efficiently (using a trace amount of a sample, and which is both simple and rapid to use) through utilization of a microsized channel has been attracting attention in a wide variety of fields including medicine, drug discovery, healthcare, environment, and food as well as biochemical research.

Among such devices, a microanalysis chip which is based on paper has an advantage in that the chip is lightweight and available at low cost in comparison to related-art devices, which eliminates a need for use of a power source, and which has high disposability.

Accordingly, the chip has been considered as a test device to be used in, for example, medical activities in developing countries and remote places where medical equipment is not prepared, disaster sites, and airports where the spread of an infectious disease needs to be prevented at the border. In addition, the chip is inexpensive and easy to handle, as allowing for use as a healthcare device that allows an individual to manage and monitor their own health conditions.

In the early 1990s, a microanalysis chip for performing the pretreatment, stirring, mixing, reaction, and detection of a sample on one chip was developed by forming a micron sized channel on glass or silicon through use of, for example, a photolithography method or a die. As a result, downsizing of a test system, an increase in analysis speed thereof, and a reduction in amount of a specimen, a reagent, or a waste liquid were achieved. The microchannel produced by using the photolithography technology had extremely high accuracy.

However, the production cost for such microchannels is expensive. The microchannel is also difficult to incinerate, and therefore has low disposability. Further, an incidental device, such as a syringe pump, is required when a test liquid is delivered into the channel, and the chip is limited to use in an environment where equipment is available, leading to the chip mainly being used in biochemical research laboratories.

The use of paper microanalysis chips was intended to solve those problems, by using an inexpensive material, such as paper or cloth, as its substrate, and capillary action of the material itself to enable the driving of a specimen or a test liquid. Accordingly, the paper chip can be used at a lower cost and in an environment without electricity. In addition, the paper chip is easy to carry (distribute) and has high disposability (combustion).

Further, maintenance of the device is not required, and unskilled operators can easily perform point of care (POC) diagnosis with the device anywhere (worldwide, with no electricity required) at low cost. Accordingly, research and development of a paper microchannel device which is intended for various infectious diseases and specific diseases, and healthcare (chronic disease management and health management) have been advanced in research institutes in the world.

A microchannel device with a hydrophilic or porous channel region which is surrounded by a hydrophobic channel wall which is provided inside or on a substrate is used as the microchannel device. In a device in which a channel is formed inside a porous substrate, high hydrophobicity is required for a material for forming the channel in order to prevent exudation of a liquid which serves as a specimen or a test liquid into the channel wall and prevent swelling of the channel wall due to water absorption when the device is used under a high humidity environment. The “channel wall” means a wall that defines a channel through which a liquid flows.

### Method of Forming Channel Wall

A thermoplastic material is generally used for forming the channel wall. U.S. Patent Application Publication No. 2012/019684 refers to an example of using fats and oils such as wax to create the channel wall. Further, an example of using a styrene-acrylic resin is shown in Japanese Patent Laid-Open No. 2022-130344.

What is common to those examples is a technology in which a thermoplastic material is thermally melted and permeated into a porous substrate, and then cooled and re-solidified within the porous substrate. The thermoplastic material can form a stable channel wall because the thermoplastic material is easily combined with the porous substrate.

### Application of Channel as Test Device

Microchannel devices include a configuration in which a specimen permeates in a thickness direction of a porous substrate (vertical flow) and a configuration in which a specimen permeates in a horizontal direction (lateral flow). A reagent for a colorimetric test or a fluorescence test is arranged in a part of the channel. Alternatively, an electrode for an electrochemical test is arranged.

A role of the channel is to guide a specimen to a reagent or an electrode at the terminal end of the channel. Further, the role is to filter a specimen in the middle of the channel or to cause the specimen to react with another reagent in the middle of the channel. In addition, a plurality of tests can be performed simultaneously by branching the channel.

In the case of a microchannel device in which a channel is formed inside a porous substrate and which has an acting section for a dispensed liquid on a back side of a dispensing section of a specimen, when a specimen is dropped on a specimen collection section on the porous substrate and the collection section is not at least a specific volume or larger, then overflows of the collection section can occur either, while the specimen is being dropped, or immediately after being dropped.

Further, when a channel is to be sufficiently filled with a specimen and a collection area is large, the required amount of the specimen may be increased.

Further, when a plurality of vertical flow porous substrate channels are laminated, leakage of the specimen can occur between the substrate channels.

### SUMMARY

The present disclosure is directed to providing an analysis device that achieves ease of dropping a specimen and a reduction in the required amount of a specimen.

The present disclosure is directed to an analysis device including a first sheet and a second sheet which is laminated below the first sheet,
the first sheet including:
   a first channel wall which is formed by filling pores of a porous substrate with a hydrophobic material; and
   a first channel which is formed by being bound by the first channel wall,
wherein, when an upper surface of the first sheet is defined as a first surface and a lower surface of the first sheet is defined as a second surface, a dispensing section through which a liquid is allowed to be dispensed is provided on the first surface of the first sheet,
wherein, when a cross-sectional area of the first channel, which is located in a direction which is parallel to the first surface and the second surface, is defined as a channel area A, the first channel has a region A which is formed so that the channel area A decreases from the first surface toward the second surface, and
wherein an acting section on which the dispensed liquid is allowed to act is present on the second sheet.

Further, the present disclosure is also directed to an analysis device including a first sheet and a second sheet which is laminated below the first sheet,
the first sheet including:
   a first channel wall which is formed by filling pores of a porous substrate with a hydrophobic material; and
   a first channel which is formed by being bound by the first channel wall,
wherein, when an upper surface of the first sheet is defined as a first surface, a lower surface of the first sheet is defined as a second surface, and a cross-sectional area of the first channel, which is located in a direction which is parallel to the first surface and the second surface, is defined as a channel area A, the first channel has a region A which is formed so that the channel area A decreases from the first surface toward the second surface,
wherein the second sheet includes:
   a second channel wall which is formed by filling pores of a porous substrate with a hydrophobic material; and
   a second channel which is formed by being bound by the second channel wall,
wherein, when an upper surface of the second sheet is defined as a third surface, a lower surface of the second sheet is defined as a fourth surface, and a cross-sectional area of the second channel, which is located in a direction which is parallel to the third surface and the fourth surface, is defined as a channel area B, the second channel has a region B which is formed so that the channel area B decreases from the third surface toward the fourth surface, and
wherein, when a dropped specimen flows in an order of the first surface, the second surface, the third surface, and the fourth surface, at an interface section between the first sheet and the second sheet,
(i) the second surface and the third surface are opposed to each other,
(ii) a region C is present in a portion in which the region A and the region B overlap each other, and
(iii) in the region C, the cross section of the first channel at the second surface is arranged so as to be included in the cross section of the second channel at the third surface.

Features of the present disclosure will become apparent from the following description of embodiments with reference to the attached drawings. The following description of embodiments is described by way of example.

### BRIEF DESCRIPTION OF THE DRAWINGS

FIG. 1A is an explanatory perspective view for schematically illustrating a first embodiment of the present disclosure.

FIG. 1B is a sectional view of FIG. 1A.

FIG. 2A is an explanatory sectional view for schematically illustrating a state before a thermoplastic resin permeates.

FIG. 2B is an explanatory sectional view for schematically illustrating a state after the thermoplastic resin permeates.

FIG. 3A is an example of an explanatory sectional view for schematically illustrating the first embodiment of the present disclosure.

FIG. 3B is an example of an explanatory sectional view for schematically illustrating a comparative example of the first embodiment of the present disclosure.

FIG. 3C is another example of the explanatory sectional view for schematically illustrating the comparative example of the first embodiment of the present disclosure.

FIG. 3D is another example of the explanatory sectional view for schematically illustrating the comparative example of the first embodiment of the present disclosure.

FIG. 4A is an explanatory perspective view for schematically illustrating a second embodiment of the present disclosure.

FIG. 4B is a sectional view of FIG. 4A.

FIG. 5 is an explanatory view for schematically illustrating collection pores and a laminate in the second embodiment of the present disclosure.

FIG. 6A is an explanatory sectional view for schematically illustrating the second embodiment of the present disclosure.

FIG. 6B is an explanatory sectional view for schematically illustrating a comparative example of the second embodiment of the present disclosure.

FIG. 7 is an explanatory sectional view for schematically illustrating a third embodiment of the present disclosure.

FIG. 8 is an explanatory perspective view for schematically illustrating a fourth embodiment of the present disclosure.

FIG. 9A is an explanatory perspective view for schematically illustrating a fifth embodiment of the present disclosure.

FIG. 9B is a sectional view of FIG. 9A.

### DESCRIPTION OF THE EMBODIMENTS

An exemplary embodiment of the present disclosure is described below with reference to the drawings. The following embodiment is illustrative, and the present disclosure is not limited to the contents of the embodiment. In addition, in the following respective drawings, constituents that are not required for the description of the embodiment are omitted from the drawings.

### First Embodiment

A first embodiment relates to an analysis device.

The analysis device of the present disclosure is an analysis device including a first sheet and a second sheet which is laminated below the first sheet,
the first sheet including:
   a first channel wall which is formed by filling pores of a porous substrate with a hydrophobic material; and
   a first channel which is formed by being bound by the first channel wall,
wherein, when an upper surface of the first sheet is defined as a first surface and a lower surface of the first sheet is defined as a second surface, a dispensing section through which a liquid is allowed to be dispensed is provided on the first surface side of the first sheet,
wherein, when a cross-sectional area of the first channel, which is located in a direction which is parallel to the first surface and the second surface, is defined as a channel area A, the first channel has a region A which is formed so that the channel area A decreases from the first surface toward the second surface, and
wherein an acting section on which the dispensed liquid is allowed to act is present on the second sheet.

In the present disclosure, an example is given in which a channel wall is formed by causing a styrene-acrylic resin to permeate into a porous substrate. First, a porous substrate and a channel wall-forming material, which are device constituent materials, are described.

### Porous Substrate

A substrate which has moderate porosity and moderate hydrophilicity is suitable as the porous substrate. For example, an open cell structure and a network (e.g., nanofiber) structure are each desired as a porous structure, and examples of a product with such structure include filter paper, plain paper, woodfree paper, watercolor paper, Kent paper, synthetic paper, a synthetic resin porous film, cloth, and a fiber product. Among those, filter paper is one exemplary material because the filter paper exhibits a high porosity and satisfactory hydrophilicity.

The porosity, which may be appropriately selected in accordance with purposes, can be from 20% to 90%. When the porosity exceeds 90%, strength as a substrate may be prevented from being maintained. When the porosity is less than 20%, permeability of a sample liquid may deteriorate.

Hydrophilicity is a property which is required for enabling a water-containing biological fluid, such as blood, urine, or saliva, which serves as the sample liquid to diffuse in the substrate.

In the analysis device of the present disclosure, thickness of the first sheet can be 0.05 mm or more and 0.5 mm or less. An average thickness of the porous substrate can be appropriately selected in accordance with the purposes, but thickness from 0.05 mm to 0.5 mm is often used. When the average thickness is less than 0.05 mm, strength as a substrate may be prevented from being maintained.

Further, a ratio of the inside of the channel wall to the entire thickness becomes larger, and hence an advantageous effect becomes difficult to obtain. However, by selecting a substrate with a small porosity or a substrate with a small fiber diameter, a similar effect can be obtained even with a thin substrate. A porous substrate with thickness from about 0.2 to about 0.5 mm is used depending on the application, in some cases, and the present disclosure is suitable for forming a channel wall in a thick porous substrate.

Parameters of the porous substrate which is used in the first embodiment are as follows:
Basis weight: 87 g/m²
Thickness: 0.17 mm
Porosity: 66%.

### Channel Forming Material

As a channel forming material in the present disclosure, a material in which wax (fats and oils) is contained in a thermoplastic resin that permeates into a porous substrate when being melted by heat is used.

The thermoplastic resin is not particularly limited, and for example, the following known thermoplastic resins can be used. Examples thereof include a polyester resin, a vinyl resin, an acrylic resin, a styrene-acrylic resin, polyethylene, polypropylene, polyolefin, an ethylene-vinyl acetate copolymer resin, and an ethylene-acrylic acid copolymer resin.

The wax (fats and oils) is also not particularly limited, and for example, the following known waxes can be used. Examples thereof include natural ester wax, hydrocarbon wax, and amide wax.

In the first embodiment, a channel forming material (toner) which contains hydrocarbon wax in a styrene-acrylic resin that easily bleeds and spreads, with a storage modulus G′ of 0.4 Pa and a loss modulus G″ of 0.1 Pa at 200° C.

### Formation Process of Channel Pattern

A method of producing an analysis device is described. A channel pattern-forming unit to which an electrophotographic printer is applied is used for channel pattern formation. A styrene-acrylic resin material is used as a developer (powder toner) which is used for the forming unit, and the material is thermally melted and permeated into a porous substrate to form a channel wall.

After passing through an electrostatic latent image formation process, a developer image formation process, and a transfer process, which are image formation processes of the electrophotographic printer, only a final fixing process is suspended. The developer image on the porous substrate onto which the developer image has been transferred is discharged to an outside of the image forming unit while keeping the developer image on an upper side in a gravity direction, and the developer image on the porous substrate is temporarily stocked (such a developer image is referred to as “unpermeated image”).

### Heating Process

The unpermeated image which carries the channel pattern passes through a heating process by a heating unit. An oven (Yamato Scientific Co., Ltd., FORCED CONVECTION OVEN DN610H) was used as the heating unit. However, a heating system is not limited thereto, and a far infrared heater, a hot plate, etc. may be used, and the heating conditions should be selected in accordance with the physical properties of the toner resin particles and the porous substrate.

When the substrate passes through the heating process, the styrene-acrylic resin particles (toner) melt to permeate into the porous substrate, which causes forming an analysis device which includes a channel surrounded by hydrophobic walls. A heating temperature needs to be set to temperature at which the toner melts to permeate the porous substrate. In configuration of the first embodiment, the toner permeated the porous substrate at 140° C. or more.

A heating time needs to be the time period for which the molten toner completely permeates in a thickness direction of the porous substrate. When the heating time is excessively long, the toner diffuses to an extent more than necessary, and hence the channel after the heating process may be narrower than the channel pattern subjected to a channel pattern image formation. In the configuration of the first embodiment, a moderate channel wall was able to be formed by setting the heating time to from 5 minutes to 30 minutes.

From the above, heating conditions in the first embodiment were set as follows: the toner was heated in an environment at 200° C. for 10 minutes. As described above, a microchannel which is surrounded by channel walls can be created in a porous substrate by combining the channel pattern image formation process and the heating process.

### Details of Heating Process and Device Production

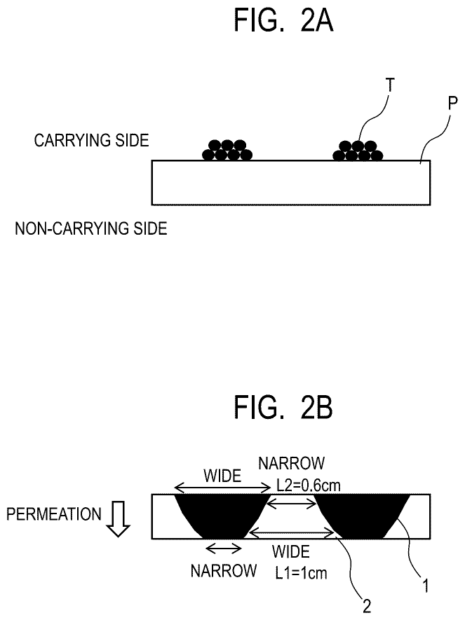

Toner T before melting in the heating process under the above conditions is placed as powder on a porous substrate P as illustrated in FIG. 2A.

When heating the powder, the toner thermally melts and permeates into the substrate. When the toner permeates into the substrate, the toner permeates in both a thickness direction and a surface direction. Accordingly, as illustrated in FIG. 2B, the toner has a vertically asymmetric shape such that a center is narrowed in a permeation direction. Meanwhile, a first channel 2 is surrounded by a first channel wall 1, and hence spreading of the channel wall on a toner-carrying side is larger than that on a non-carrying side. Accordingly, a channel area is narrower on the toner-carrying side than on the non-carrying side.

In the first embodiment, L2 was 0.6 cm with respect to L1 of 1 cm, which was smaller by 0.4 cm.

Production of a device with the channel pattern as described above is described. In the analysis device of the present disclosure, when an upper surface of the first sheet is defined as a first surface and a lower surface of the first sheet is defined as a second surface, a dispensing section through which a liquid is allowed to be dispensed is provided on the first surface side of the first sheet. An area of a cross section of the first channel, which is located in a direction which is parallel to the first surface and the second surface, is defined as a channel area A, and the first channel has a region A formed such that the channel area A decreases from the first surface toward the second surface. An acting section on which the dispensed liquid is allowed to act is present on the second sheet.

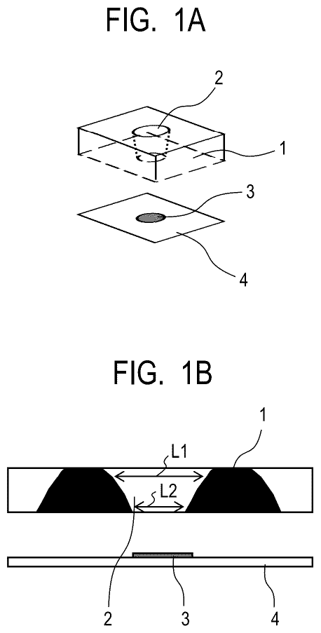

FIG. 1A is a perspective view of the first embodiment, and FIG. 1B is a sectional view of the first embodiment, which uses the same reference numbers as FIG. 1A. The analysis device of the present disclosure is an analysis device which includes a first sheet and a second sheet which is laminated below the first sheet. The first sheet includes: the first channel wall 1 which is formed by filling pores of a porous substrate with a hydrophobic material; and the first channel 2 which is formed by being bound by the first channel wall 1. A PET sheet is used as the second sheet, and the first sheet and the second sheet are bonded to each other with a double-sided tape (not shown). An adhesive layer is a hydrophobic acrylic or rubber adhesive.

In the analysis device of the present disclosure, the first sheet can be a vertical flow sheet. For details, a vertical channel is used for the first channel 2, which a side with a larger channel area is used as a specimen collection area. A configuration is selected in which an area of a terminal end of the channel is smaller than an area of a starting end of the channel. A reaction reagent 3 (acting section) is arranged on the adhesive layer of the second sheet (PET sheet 4) so as to be opposed to the terminal end.

As described above, in the analysis device of the present disclosure, the acting section can be present at an opposing portion of the second sheet that is opposed to the region A in the first sheet at an interface section between the first sheet and the second sheet. The reaction reagent (acting section) 3 is fixed onto the PET sheet 4, and the PET sheet also has a function as a supporting member for the porous substrate at the same time. Positivity or negativity for a specimen that has reacted with the reagent is determined by a colorimetric test, etc.

### (Comparative Study)

A ratio of the channel area A on the second surface to the channel area A on the first surface can be 25 area % or more and 80 area % or less. The channel area A on the first surface can be 0.03 cm² or more and 80 cm² or less. In these regards, an advantage of the first embodiment is described by comparing three types of channels with each other.

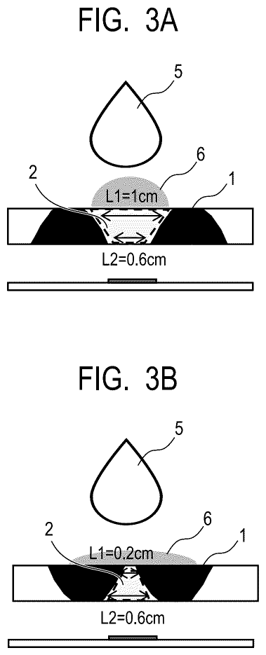

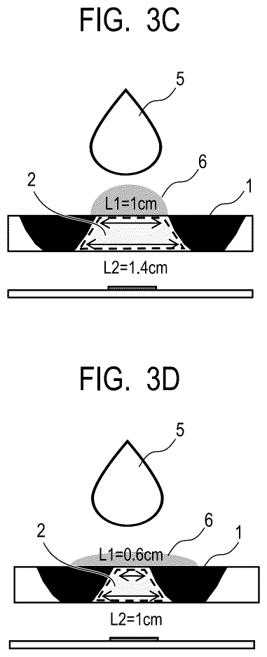

FIG. 3A illustrates the shape of the first embodiment itself. Dropping was performed on a surface with a larger channel area, and a reaction reagent of same size as the channel area at the terminal end of the channel was arranged at the terminal end. L1 was set to 1 cm, and L2 was set to 0.6 cm.

In FIG. 3B, a specimen was dropped on a surface with a smaller channel area, and a reaction reagent of same size as the channel area at the terminal end of the channel was arranged at the terminal end. L1 was set to 1 cm, and L2 was set to 0.6 cm.

In FIG. 3C, a specimen was dropped on a surface with a smaller channel area. An area of the starting end of the channel was set to same size as that in FIG. 3A. L1 was set to 1 cm, and L2 was set to 1.4 cm.

FIG. 3D is a vertically inverted version of FIG. 3A, in which L1 was set to 0.6 cm and L2 was set to 1 cm.

Compared to FIG. 3A, collection areas which are shown in FIG. 3B and FIG. 3D are smaller as compared to those in FIG. 3A, and dropping of a specimen 5 needs to be performed for a longer time. In FIG. 3B, when a dropping speed is high, a specimen 6 may overflow after dropping. An area of L1=1 cm in FIG. 3A can be determined to be a collection area in which dropping can be easily accomplished.

FIG. 3C has same collection area as that in FIG. 3A, and can thus be determined to be a sufficient collection area for dropping. However, when a specimen is dropped on a surface with a smaller channel area while maintaining sufficient L1, the required amount of the specimen to fill the channel increases. A volume in the first channel 2 can be approximated by a shaded truncated cone in which diameters of a bottom surface and an upper surface are different from each other. The amount of the specimen for each of those configurations was calculated, and the results were summarized.

TABLE 1

| | | Required amount | |
|---|---|---|---|
| | Volume | of specimen | Ease of |
| | ratio | μL | dropping |
| FIG. 3A | 1 | 100 | ○ |
| FIG. 3B | 0.25 | 25 | × |
| FIG. 3C | 2.25 | 225 | ○ |
| FIG. 3D | 1 | 100 | × |

Based on the results for FIG. 3B and FIG. 3D in Table 1, dropping of the specimen becomes difficult for these configurations. According to the result for FIG. 3C in Table 1, there is a larger required amount of the specimen.

Therefore, when the analysis device has a channel area that narrows from a specimen collection side (channel starting end) to a channel terminal end, while ensuring at least a certain area for the collection area (channel starting end), the required amount of the specimen is smaller.

In the first embodiment, with a use of a sample in which the channel area becomes narrower from the starting end side moving toward the terminal end side (as illustrated in FIG. 1A and FIG. 1B), both ease of dropping a specimen and a reduction in the required amount of the specimen were able to be achieved.

In the present disclosure, the size of the channel area that can be considered an effective size can be any channel diameter, provided openings (L1, L2, L3, etc.) of the channel satisfy L=0.098 cm to 3 cm (corresponding to from 0.03 cm² to 80 cm² in area), and an area ratio of the starting end to the terminal end falls within a range from 25% to 80% (in this embodiment, L1=1 cm and L2=0.6 cm (36% in area ratio)).

### Second Embodiment

The second embodiment relates to an example in which vertical channels are laminated. An analysis device of the present disclosure includes a first sheet and a second sheet which is laminated below the first sheet,
the first sheet including:
   a first channel wall which is formed by filling pores of a porous substrate with a hydrophobic material; and
   a first channel which is formed by being bound by the first channel wall,
wherein, when an upper surface of the first sheet is defined as a first surface, a lower surface of the first sheet is defined as a second surface, and a cross-sectional area of the first channel, which is located in a direction which is parallel to the first surface and the second surface, is defined as a channel area A, the first channel has a region A which is formed so that the channel area A decreases from the first surface toward the second surface,
wherein the second sheet includes:
   a second channel wall which is formed by filling pores of a porous substrate with a hydrophobic material; and
   a second channel which is formed by being bound by the second channel wall,
wherein, when an upper surface of the second sheet is defined as a third surface, a lower surface of the second sheet is defined as a fourth surface, and a cross-sectional area of the second channel which is located in a direction which is parallel to the third surface and the fourth surface, is defined as a channel area B, the second channel has a region B which is formed so that the channel area B decreases from the third surface toward the fourth surface, and
wherein, when a dropped specimen flows in an order of the first surface, the second surface, the third surface, and the fourth surface, at an interface section between the first sheet and the second sheet,
(i) the second surface and the third surface are opposed to each other,
(ii) a region C is present in a portion in which the region A and the region B overlap each other, and
(iii) in the region C, the cross section of the first channel at the second surface is arranged so as to be included in the cross section of the second channel at the third surface.

Descriptions of items in the second embodiment which are same with those in the first embodiment may be omitted below.

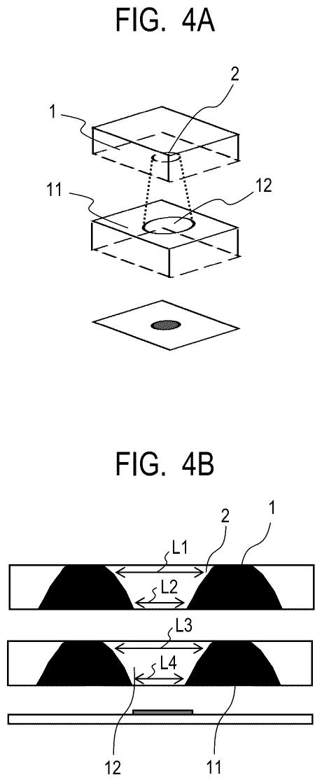

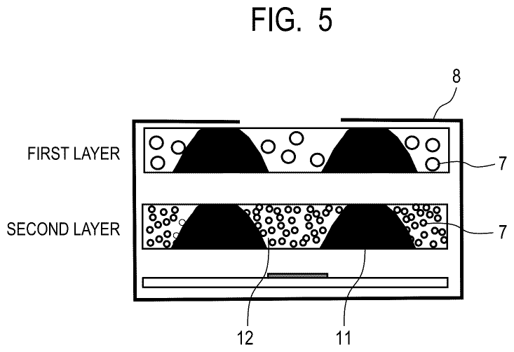

FIG. 4A is a perspective view of the analysis device of the second embodiment, and FIG. 4B is a sectional view thereof. The purpose of lamination is to provide the porous substrate with a filtering function. For that purpose, in the analysis device of the present disclosure, a collection particle diameter of the second channel can be smaller than a collection particle diameter of the first channel. In view of the foregoing, collection pores 7 were changed for each layer as illustrated in FIG. 5. For example, by a collection particle diameter of the first layer (first sheet) being set to 20 μm and a collection particle diameter of the second layer (second sheet) being set to 10 μm, particles can be collected in order from larger particles while suppressing inhibition of a flow rate.

In the analysis device of the present disclosure, the second sheet includes: a second channel wall 11 formed by filling pores of a porous substrate with a hydrophobic material; and a second channel 12 formed by being bound by the second channel wall 11. An upper surface of the second sheet is defined as a third surface and a lower surface of the second sheet is defined as a fourth surface, and a cross-sectional area, of the second channel 12, which is located in a direction which is parallel to the third surface and the fourth surface, is defined as a channel area B, such that the second channel 12 includes a region B which is formed so that the channel area B decreases from the third surface toward the fourth surface.

Similarly to the first embodiment, after channel patterns of the first layer and the second layer are formed and then heated, the channel pattern of the first layer, the channel pattern of the second layer, and a PET sheet 4 with an acting section 3 are integrated and fixed by being covered with a laminate 8 in FIG. 5.

The second sheet and the PET sheet 4 are bonded to each other with a double-sided tape (not shown). An adhesive layer is a hydrophobic acrylic or rubber adhesive. The first sheet and the second sheet are also bonded to each other with a double-sided tape (not shown). However, the first channel and the second channel need to be connected to each other at least in the region C, and hence, a portion without adhesive layer is provided in an area larger than the region C between the first sheet and the second sheet. Accordingly, when a plurality of vertical flow porous substrate channels are laminated, leakage of a specimen may occur between the substrates.

### Comparative Study

A channel diameter at a starting end of the first layer is defined as L1, a channel diameter at a terminal end of the first layer is defined as L2, a channel diameter at a starting end of the second layer is defined as L3, and a channel diameter at a terminal end of the second layer is defined as L4.

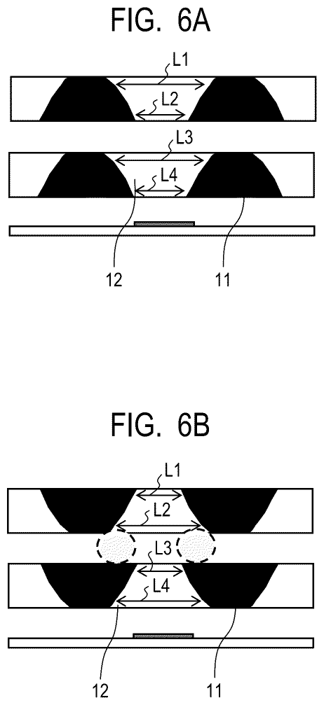

A device with a shape of L1=1.0 cm, L2-0.6 cm, L3=1.0 cm, and L4-0.6 cm as illustrated in FIG. 6A is defined as the second embodiment. At a boundary between the first layer and the second layer, the device is configured such that a relationship of L2<L3 is satisfied. In addition, the device is configured such that the channel area region of the first layer is included in the channel area region of the second layer.

In the analysis device of the present disclosure, when a dropped specimen flows in an order of a first surface, a second surface, a third surface, and a fourth surface, at an interface section between a first sheet and a second sheet,
(i) the second surface and the third surface are opposed to each other,
(ii) a region C is present in a portion in which a region A and a region B overlap each other, and
(iii) in the region C, the cross section of the first channel at the second surface is arranged so as to be included in the cross section of the second channel at the third surface.

The “region C” in the present disclosure refers to, for example, a boundary region between the first layer and the second layer.

A device with a shape of L1=0.6 cm, L2=1.0 cm, L3=0.6 cm, and L4=1.0 cm as illustrated in FIG. 6B is defined as a comparative example. The device is configured such that, at a boundary between the first layer and the second layer, a relationship of L2>L3 is satisfied. In addition, the device is configured such that at least a part of the channel area region of the first layer is not included in the channel area region of the second layer.

The two types of devices which are described above were prepared, and whether leakage of a specimen which is occurred at a boundary surface or not was examined by focusing on the relationship between L2 and L3. The results are as follows.

TABLE 2

| | Results |
|---|---|
| Embodiment | Specimen leaks |
| Comparative Example | Specimen does not |
| | leak |

Specimen leakage occurred starting from a position which is indicated by a dotted line in FIG. 6B.

From those results, the starting end of the second layer needs to be wider than the terminal end of the first layer. When a specimen comes into contact with the hydrophilic channel of the second layer, the specimen is absorbed from the contact portion, and thus does not leak. When the terminal end of the first layer comes into contact with a portion of the hydrophobic channel wall of the second layer, the contact may generate a risk that a microscopic gap is thus generated, resulting in leakage. Accordingly, the starting end of the second layer needs to be widely provided so as to encompass the terminal end of the first layer, in other words, needs to cover the terminal end of the first layer so that the terminal end of the first layer is included in the starting end of the second layer.

In this embodiment, as illustrated in FIG. 5, when the device is configured such that channel area of terminal end of first layer<channel area of starting end of second layer is satisfied and the terminal end of the first layer is included in the starting end of the second layer, leakage between the laminated layers can be reduced. Further, the first embodiment and this embodiment can be simultaneously established. Even with a laminated device, while both ease of dropping a specimen and a reduction in the specimen amount can be achieved, leakage from between the laminated layers can also be reduced.

### Third Embodiment

This embodiment provides another purpose of laminating vertical channels and demonstrates a magnitude relationship of the channel areas in this case. In addition to the configuration of the second embodiment, the analysis device of the present disclosure can have a configuration in which at least one selected from the group which consists of a first channel and a second channel has a function of reacting with a reagent.

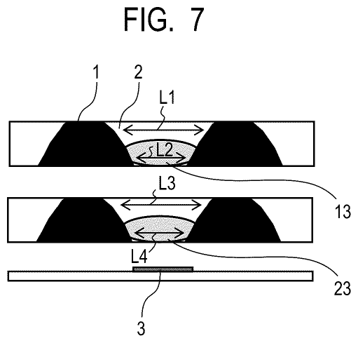

As illustrated in FIG. 7, a configuration is also conceivable in which a reaction reagent 13 and a reaction reagent 23 are arranged in vertical channels. Contact with a plurality of reagents is assumed to be performed in stages. In order to reliably provide an opportunity of contact with a reagent which exhibits a certain concentration or a concentration higher than the certain concentration, to arrange the reagent on a terminal end side of the channel and to set the terminal end side (L2) narrower than a starting end side (L1) as illustrated in FIG. 7 is desirable. In short, the device is configured such that channel area of starting end (L1)>channel area of terminal end (L2) is satisfied in the corresponding layer.

Further, similarly to the second embodiment, the device is configured such that a relationship of channel area of terminal end of first layer (L2)<channel area of starting end of second layer (L3) is satisfied for a purpose of reducing leakage at a connection portion between the first layer and the second layer.

### Fourth Embodiment

In addition to the configuration of the first embodiment, the analysis device of the present disclosure can have a configuration in which the second sheet includes: a third channel wall 21 which is formed by filling pores of a porous substrate with a hydrophobic material; and a third channel 22 which is formed by being bound by the third channel wall 21, and the second sheet is a lateral flow sheet.

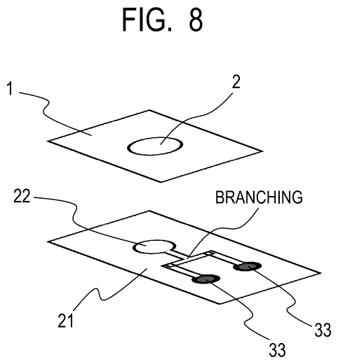

In this embodiment, a purpose of laminating vertical channels and lateral channels in combination is shown with reference to FIG. 8, and a magnitude relationship of the channel areas in this case is shown. A channel of the first layer (first sheet) is configured to include a filtering function.

Similar to the second embodiment, a relationship of channel area of terminal end of first layer<channel area of starting end of second layer is satisfied for the purpose of reducing leakage at a connection portion between the first layer and the second layer (second sheet). The second layer is a lateral flow layer, and the purpose is to branch a specimen into a plurality of routes. Different reagents are arranged at terminal ends of the second layer, and perform desired reactions with the specimen.

As in the second embodiment, two vertical flow sheets may be prepared, and a lateral flow sheet may be prepared below the two vertical flow sheets. In this case, in the analysis device of the present disclosure, a third sheet can be laminated below the second sheet, and the third sheet can include: a third channel wall formed by filling pores of a porous substrate with a hydrophobic material; and a third channel formed by being bound by the third channel wall, and be a lateral flow sheet.

### Fifth Embodiment

In addition to the configuration of the first embodiment, the analysis device of the present disclosure can have a configuration in which
a fourth sheet is laminated between a first sheet and a second sheet,
the fourth sheet includes: a second channel wall 11 which is formed by filling pores of a porous substrate with a hydrophobic material; and a second channel 12 and a fourth channel 32 which are formed by being bound by the second channel wall 11,
the second sheet includes: a fourth channel wall 31 which is formed by filling pores of a porous substrate with a hydrophobic material; and a fifth channel 44 and a sixth channel 45 which are formed by being bound by the fourth channel wall 31,
the second channel 12 and the fourth channel 32 are connected to each other via a seventh channel 46, and
acting sections 43 on each of which the dispensed liquid is allowed to act are present in the fifth channel 44 and the sixth channel 45, respectively.

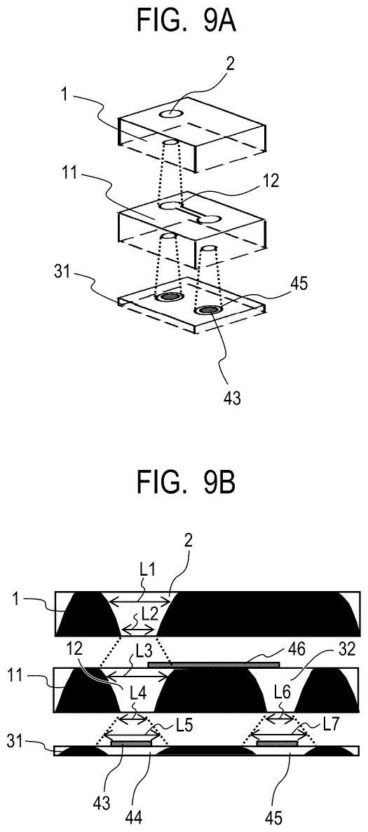

This embodiment provides another example in which vertical channels are laminated. Reference is made to FIG. 9A and FIG. 9B. FIG. 9A is a perspective view, and FIG. 9B is a sectional view. This channel is a channel such that a lateral channel is combined with the fourth sheet (second layer) to branch the channel to thereby allow simultaneous reaction with a plurality of reaction reagents 33.

In this embodiment, independently of achieving both a reduction in the required amount of specimen and ease of dropping a specimen by size of the area in the vertical channel as in the first embodiment, the size of the area of the opposing surfaces between layers is determined from a viewpoint whether leakage occurs or not.

In the second layer, channels are arranged so as to overlap each other on a surface in order to cause a specimen to flow in a lateral direction. Further, a layer of a reaction reagent is set as a third layer, a periphery of the reagent is surrounded by a channel, and a periphery of the channel is further surrounded by a channel wall. This is for reducing leakage of a specimen between the second layer and the third layer.

In this embodiment, opposing surfaces between layers are present at three locations. In the first location, a relationship of L2=0.6 cm and L3=1.0 cm is satisfied, and L2 is set smaller so as to be included in L3 (L2<L3) similarly to the second embodiment.

In the present disclosure, the first layer corresponds to the first sheet, and the second layer corresponds to the fourth sheet. In the second location, a range surrounded by the channel of the third layer is defined as L5, and a relationship of L4=0.6 cm and L5=1.0 cm is satisfied. L4 is set smaller so as to be included in L5 (L4<L5).

In the present disclosure, the second layer corresponds to the fourth sheet, and the third layer corresponds to the second sheet. The periphery of the reaction reagent is surrounded by the channel, and is thus hydrophilic, which results that the specimen stays in this region of L5. In the third location, a terminal end of the second layer which is subsequent to the lateral channel was set as L6-0.6 cm, and a starting end of the third layer was set as L7=1.0.

In this case, L6 is set smaller so as to be included in L7 (L6<L7). In the present disclosure, the second layer corresponds to the fourth sheet, and the third layer corresponds to the second sheet. With the configuration as described above, leakage of the specimen between the layers was able to be reduced.

In the present disclosure, by setting the channel area at the terminal end to be narrower than the channel area at the specimen collection surface (starting end) of the device, improvement in ease of dropping a specimen and a reduction in the required amount of the specimen can be achieved.

Further, in the present disclosure, when a plurality of porous substrates is laminated, by setting a channel area range of a terminal end of a preceding substrate to be widely provided so that a channel area range of a starting end of a subsequent substrate encompasses the channel area range of the terminal end of the preceding substrate, leakage of a specimen can be reduced.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-033813, filed Mar. 4, 2025, which is hereby incorporated by reference herein in its entirety.

What is claimed is:

1. An analysis device comprising a first sheet and a second sheet which is laminated below the first sheet,
the first sheet comprising:
a first channel wall which is formed by filling pores of a porous substrate with a hydrophobic material; and
a first channel which is formed by being bound by the first channel wall,
wherein, an upper surface of the first sheet is defined as a first surface and a lower surface of the first sheet is defined as a second surface, and a dispensing section through which a liquid is allowed to be dispensed is provided on the first surface of the first sheet,
wherein, a cross-sectional area of the first channel, which is located in a direction parallel to the first surface and the second surface, is defined as a channel area A, the first channel has a region A which is formed so that the channel area A decreases from the first surface toward the second surface, and
wherein an acting section on which the dispensed liquid is allowed to act is present on the second sheet.

2. The analysis device according to claim 1, wherein a ratio of the channel area A on the second surface to the channel area A on the first surface is 25 area % or more and 80 area % or less.

3. An analysis device comprising a first sheet and a second sheet which is laminated below the first sheet,
the first sheet comprising:
a first channel wall which is formed by filling pores of a porous substrate with a hydrophobic material; and
a first channel which is formed by being bound by the first channel wall,
wherein, an upper surface of the first sheet is defined as a first surface, a lower surface of the first sheet is defined as a second surface, and a cross-sectional area of the first channel, which is located in a direction which is parallel to the first surface and the second surface, is defined as a channel area A, and the first channel has a region A which is formed so that the channel area A decreases from the first surface toward the second surface,
wherein the second sheet comprises:
a second channel wall which is formed by filling pores of a porous substrate with a hydrophobic material; and
a second channel which is formed by being bound by the second channel wall,
wherein, an upper surface of the second sheet is defined as a third surface, a lower surface of the second sheet is defined as a fourth surface, and a cross-sectional area of the second channel, which is located in a direction which is parallel to the third surface and the fourth surface, is defined as a channel area B, and the second channel has a region B which is formed so that the channel area B decreases from the third surface toward the fourth surface, and
wherein, a dropped specimen flows in order from the first surface, the second surface, the third surface, and the fourth surface, at an interface section between the first sheet and the second sheet,
(i) the second surface and the third surface are opposed to each other,
(ii) a region C is present in a portion in which the region A and the region B overlap each other, and
(iii) in the region C, the cross section of the first channel at the second surface is arranged so as to be included in the cross section of the second channel at the third surface.

4. The analysis device according to claim 3, wherein a collection particle diameter of the second channel is smaller than a collection particle diameter of the first channel.

5. The analysis device according to claim 3, wherein at least one selected from the group consisting of the first channel and the second channel has a function of reacting with a reagent.

6. The analysis device according to claim 1, wherein the acting section is present at an opposing portion of the second sheet that is opposed to the region A in the first sheet, at an interface section between the first sheet and the second sheet.

7. The analysis device according to claim 1, wherein the second sheet comprises:
a third channel wall which is formed by filling pores of a porous substrate with a hydrophobic material; and
a third channel which is formed by being bound by the third channel wall,
wherein the second sheet is a lateral flow sheet.

8. The analysis device according to claim 3, further comprising a third sheet which is laminated below the second sheet,
wherein the third sheet comprises:
a third channel wall which is formed by filling pores of a porous substrate with a hydrophobic material; and
a third channel which is formed by being bound by the third channel wall,
wherein the third sheet is a lateral flow sheet.

9. The analysis device according to claim 1, wherein the first sheet is a vertical flow sheet.

10. The analysis device according to claim 3, wherein the first sheet is a vertical flow sheet.

11. The analysis device according to claim 1,
wherein the channel area A on the first surface is 0.03 cm² or more and 80 cm² or less, and
wherein thickness of the first sheet is 0.05 mm or more and 0.5 mm or less.

12. The analysis device according to claim 3,
wherein the channel area A on the first surface is 0.03 cm² or more and 80 cm² or less, and
wherein thickness of the first sheet is 0.05 mm or more and 0.5 mm or less.
